(19) United States
(12) Patent Application Publication
(10) Pub. No.: US 2016/0029579 A1
(43) Pub. Date: Feb. 4, 2016

(54) Modular Algal Aquaculture System and Method

(71) Applicant: SABRTECH INC., Herring Cove (CA)

(72) Inventors: W. Mather A. CARSCALLEN, Herring Cove (CA); William E. CARSCALLEN, Ottawa (CA); Christopher P. KOZELA, Herring Cove (CA); Charlotte UNDERWOOD, Quebec (CA)

(73) Assignee: Sabrtech Inc., Nova Scotia (CA)

(21) Appl. No.: 14/774,001

(22) PCT Filed: Mar. 13, 2014
(86) PCT No.: PCT/CA2014/050227

§ 371 Date: Sep. 9, 2015

Related U.S. Application Data
(60) Provisional application No. 61/781,419, filed on Mar. 14, 2013.

(51) Int. Cl. A01G 31/02; A01G 7/04; A01G 1/00; A01G 33/00

(52) U.S. Cl. CPC A01G 31/02; A01G 33/00; A01G 7/045; A01G 1/001

(57) ABSTRACT

The present invention provides an improved modular algal aquaculture system and method including a modular algal aquaculture component having a cultivation surface having a first end and a second end, a supply header located adjacent to one of the first end and the second end, a return header located adjacent to the other of the first end and the second end, a reservoir, the reservoir in fluid communication with the supply header and the return header, a pump, the pump in fluid communication with the reservoir and at least one of the supply header and the return header.

### FIELD

The present invention relates to systems for cultivating algae populations for use in a variety of industrial processes. More specifically, the present invention relates to an improved modular algal aquaculture system and method that can be employed in a wide variety of applications to enable cultivation and harvesting of algae in an environmentally sound and economically viable manner.

### BACKGROUND

Although there is no clear definition of ‘algae’, it can be understood to include a vast group of simple organisms that are typically found in aquatic environments. Algae are typically photosynthetic and can be unicellular or multi-cellular.

There are a number of aquacultural methods that have been developed for cultivating algae for use in a wide variety of industrial processes. One example is the use of algae to produce biofuels for use as a fossil fuel replacement or additive, however a wide variety of other uses for algae exist including use as agricultural fertilizer, livestock and aquaculture food sources, pharmaceutical preparations and nutritional supplements, among other uses for algae that will be readily understood by the skilled person.

However, there are a number of drawbacks with known algal aquaculture methods. For example, raceway and open pond systems have a large geographic foot print, can be easily contaminated by unwanted organisms, have high harvesting costs and produce relatively low amounts of algal biofuels when compared to the required inputs of energy and cost.

By way of another example, photobioreactor systems are highly complex systems that are difficult to scale up, have high harvesting costs, and require significant capital and operating costs.

Heterotrophic bioreactor systems require large amounts of feedstock, must be located near an abundant feedstock source, can be easily contaminated, do not allow sequestration of CO₂ and have high harvesting costs.

Therefore, there is need for an improved modular algal aquaculture system and method that can be employed in a wide variety of applications, is resistant to contamination, has a relatively small geographic footprint and enables cultivation and harvesting of algae in an environmentally sound and economically viable manner.

### BRIEF SUMMARY

The present invention provides an improved modular algal aquaculture system and method that can be employed in a wide variety of applications to enable cultivation and harvesting of microalgae in an environmentally sound and economically viable manner.

In at least one embodiment, the present invention provides a modular algal aquaculture component having a cultivation surface having a first end and a second end, a supply header located adjacent to one of the first end and the second end, a return header located adjacent to the other of the first end and the second end, a reservoir, the reservoir in fluid communication with the supply header and the return header, and a pump, the pump in fluid communication with the reservoir and at least one of the supply header and the return header.

In at least one embodiment, the present invention provides a modular algal aquaculture system comprising a plurality of modular algal aquaculture components, each of the modular algal aquaculture components having a cultivation surface having a first end and a second end, a supply header located adjacent to one of the first end and the second end, a return header located adjacent to the other of the first end and the second end, a reservoir, the reservoir in fluid communication with the supply header and the return header, and pump, the pump in fluid communication with the reservoir and at least one of the supply header and the return header such that the return header of a first modular algal aquaculture component fluidly communicates with at least one of the return header and the supply header of a second modular algal aquaculture component and wherein the supply header of a first modular algal aquaculture component fluidly communicates with at least one of the return header and the supply header of a second modular algal aquaculture component.

### BRIEF DESCRIPTION OF THE FIGURES

The present invention will be better understood in connection with the following figures, in which:

FIG. 1 is an isometric view of a modular algal aquaculture system component in accordance with at least one embodiment of the present invention;

FIG. 2 is a conceptual view of a modular algal aquaculture system in accordance with at least one embodiment of the present invention;

FIG. 3 is another conceptual view of multiple modular algal aquaculture systems in accordance with at least one embodiment of the present invention; and,

FIG. 4 is yet another conceptual view of multiple algal aquaculture systems in accordance with at least one embodiment of the present invention.

### DETAILED DESCRIPTION OF THE EMBODIMENTS

The present invention is a modular algal aquaculture system, methods and components therefor having a cultivation surface having a first end and a second end, a supply header located adjacent to one of the first end and the second end, a return header located adjacent to the other of the first end and the second end, a reservoir, the reservoir in fluid communication with the supply header and the return header and a pump, the pump in fluid communication with the reservoir and at least one of the supply header and the return header.

It will be readily understood that the present invention can be used in connection with any strain of suitable algae including but not limited to cyanobacteria, microalgae, macroalgae, mixed strain algal cultures, single strain algal cultures, diatoms, phototrophic algae, mixotrophic algae, among many other arrangements that will be readily understood by the skilled person. In some embodiments, it is contemplated that the algal strain selected will be determined by the geographic location, available water quality and resultant chemical profile that will vary depending on the needs of a particular application.

The present invention is contemplated as a modular system of interchangeable, interfitting and cooperating components that can be assembled in a wide variety of ways to suit the needs of a particular application. In this way the present invention is well suited to scaling up or scaling down and can be quickly assembled and disassembled as required by the needs of a particular application.

It is contemplated that each component has a cultivation surface that can be manufactured from any number of materials, such as but not limited to rubber, plastic, PVC, carbon fibre, poured concrete, steel, wood, among any other suitable materials that will be readily understood by the skilled person. In some embodiments, it is contemplated that the cultivation surface will consist of an underlying support surface and a covering surface. In other embodiments, it is contemplated that the cultivation surface is a single unitary component. It is contemplated that the cultivation surface can be an open runway or alternatively a flat surface immersed in a stream of water.

It is contemplated that the cultivation surface can be smooth or alternatively can be stippled. For the purposes of the present disclosure it is contemplated that the term “stippled” includes ridged, rippled, wavy, perforated, embossed, roughly finished and any other non-smooth surfaces. Moreover, it is contemplated that the cultivation can take any profile shape including but not limited to convex, flat and concave, and can also take any plan shape, including but not limited to rectangular, polygonal, circular, elliptical and triangular.

In at least one embodiment, it is contemplated that the cultivation surface has an upper surface where algae is cultivated, while in other embodiments, it is contemplated that the cultivation surface has a lower surface where algae is cultivated. In these latter embodiments, it is further contemplated that the cultivation surface can be perforated, so that water can be applied to the upper surface of the cultivation surface, flow through the perforated surface, and the algae can be grown from the lower surface of the cultivation surface in hanging mats.

In some embodiments it is further contemplated that the cultivation surface can include an additional adherence film that can be securely yet removably fixed to the cultivation surface. In this way the adherence film can be removed at the time of harvesting to result in more thorough retrieval of all algae produced by the present invention. Further, adherence films can be inoculated with particular algal strains that suit a particular application or alternatively can be customized to encourage particularly selected strains of algae.

In at least one embodiment, the present invention further includes a harvesting system that can manually or automatically harvest the cultivated algae from the cultivation surface. In one embodiment it is contemplated that the harvesting system is a laterally extending scraping mechanism that moves across the cultivation surface to physically dislodge or ‘scrape’ the cultivated algae from the cultivation surface, while in other embodiments it is contemplated that the harvesting system can be a vacuum mechanism that vacuums the cultivated algae from the cultivation surface, among other arrangements that will be readily appreciated by the skilled person.

It is contemplated that the present invention has a supply header for providing water to the cultivation surface from a reservoir and a return header for returning water from the cultivation surface to the reservoir. It is contemplated that the term water can include, but is not limited to, distilled water, seawater, ground water, treated water, aerated water, well water, untreated water, fresh water and salt water, among other types of water that will be readily understood by the skilled person. Further it is contemplated that the water can include nutrients, fertilizers, cleaning agents, algal cultures, dissolved wastes and solid wastes.

It is contemplated that the supply header is located adjacent one side of the cultivation surface and the return header is located adjacent another side of the cultivation surface. In at least one embodiment, the supply header is located on an opposing side of the return header, however other arrangements are also contemplated.

It is contemplated that each of the return header and the supply header can have at least one orifice. In some embodiments, the supply header is a pipe having a plurality of laterally and radially oriented orifices for supplying water to the cultivation surface from the reservoir, however other arrangements are also contemplated. For example, the supply header can be an open sluiceway or trough having an orifice that is a drain, weir or open ended trough, among other arrangements that will be readily understood by the skilled person.

In some embodiments, the return header can also be an open sluiceway or trough having an orifice that is a drain, weir or open ended trough for returning water to the reservoir, among other arrangements that will be readily understood by the skilled person. It is contemplated that the orifice of the supply header and the return header can be a weir or open-ended trough that allows water to flow onto the cultivation surface (in the case of the supply header) and off of the cultivation surface (in the case of the return header).

It is contemplated that the reservoir can be arranged in a number of ways. In one embodiment, the reservoir directly and fluidly communicates with the supply header and the return header by way of any suitable tubing or piping, including but not limited to ABS pipe, PVC pipe, copper, steel or other piping, rubber or silicone tubing, among any other suitable fluid communication arrangement that will be readily understood by the skilled person.

In some arrangements, multiple system components can be serviced by a single, common system reservoir, while in other embodiments a dedicated reservoir is provided for each system component. In this way, multiple interfitting components can be arranged to produce a modular system that can be scaled according to the requirements of a particular application.

It is contemplated that in some embodiments, the return header of a first system component is in fluid communication (or even in common) with a supply header of a second component. In this way, water that is returned from a first cultivation surface can be provided to the supply header of a second system component, in order to create compact overall systems that have low geographic footprint for the amount of biomass produced.

It is contemplated that the present invention includes a pump for moving fluid about the system from the reservoir to the supply header and/or the return header. It is contemplated that this pump could be a supply pump in communication with the reservoir and supply header or alternatively it is contemplated that the present invention could be a gravity-fed system where the reservoir is located at a higher elevation than the remainder from the system and gravity supplies head pressure to the supply header and the pump is a return pump provided to pump fluid back to the reservoir from the return header. It is contemplated that the pump can be any suitable hydraulic pump that will be readily understood by the skilled person.

In some embodiments it is contemplated that the cultivation surface is oriented at an inclined angle to horizontal such that water can flow from the outlet header to the return header under the force of gravity, as will be readily understood by the skilled person. In some embodiments, it is contemplated that that the inclination of the cultivation surface can be adjusted from horizontal to vertical as required by the instant application of the present invention.

It is further contemplated that the present invention can include a variety of optional additional systems depending on the needs of the instant application, including but not limited to, a lighting system positioned adjacent the cultivation surface, an aeration system for aerating the supply water, a heater for heating the water, a feedstock supply unit in fluid communication with the supply header, the return header or the reservoir, a sterilization unit in fluid communication with the supply header, the return header or the reservoir, a mechanical harvesting system for harvesting the cultivated algae for the cultivation surface and adjustment means for adjusting the inclination of the cultivation surface.

It is also contemplated that the present invention can be entirely contained within an enclosure (such as a standard shipping container) or alternatively it is contemplated that the present invention can be installed in a large structure (such as a greenhouse) or alternatively can be installed out of doors.

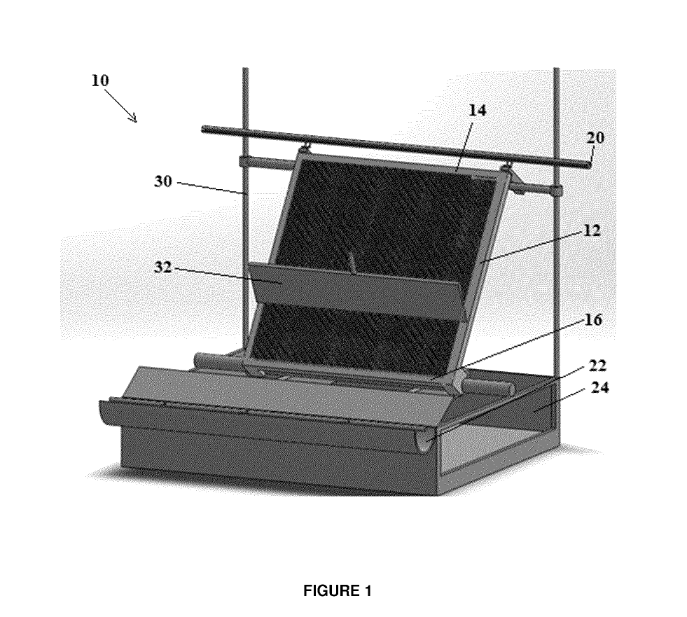

Turning to FIG. 1, at least one embodiment of a modular algal aquaculture component is illustrated. In this embodiment, modular algal aquaculture component 10 includes a cultivation surface 12 having a first end 14 and a second end 16. A supply header 20 is located adjacent first end 14 of cultivation surface 12 and a return header 22 is located adjacent second end 22 of cultivation surface 12. Both supply header 20 and return header 22 include at least one orifice (not shown) that permits fluid communication with a reservoir 24.

In this embodiment, modular algal aquaculture component 10 includes an adjustment system 30 for adjusting the inclination of the cultivation surface 12 and a mechanical harvesting system 32 that is a scraping system adapted to scrape the cultivated algae from the cultivation surface 12.

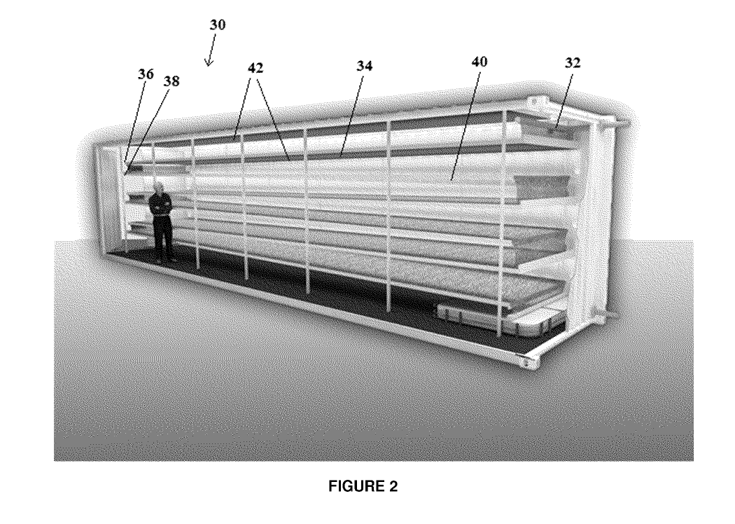

Turning to FIG. 2, at least one embodiment of a modular algal aquaculture system is illustrated. In this embodiment, multiple modular algal aquaculture components are joined together in a multi-tiered stackable arrangement 30. In this embodiment, it is contemplated that a common reservoir (not shown) is provided and that water is provided from the common reservoir to a first supply header 32 having orifices for providing water to a first cultivation surface 34, which is an open, sloped raceway. In this embodiment, the first return header 36 and associated orifice is an open trough that allows water to flow from the first cultivation surface 34 to a second supply header 38 that is an open trough. Next, water from the supply header 38 then flows to a second cultivation surface 40 that is another open, sloped raceway. It is further contemplated that a lighting system 42 can be provided on the undersurface of the cultivation surface.

In this way, multiple system components can be stacked in a closed environment to create an algal aquaculture system that has a very small low geographic footprint for biomass produced.

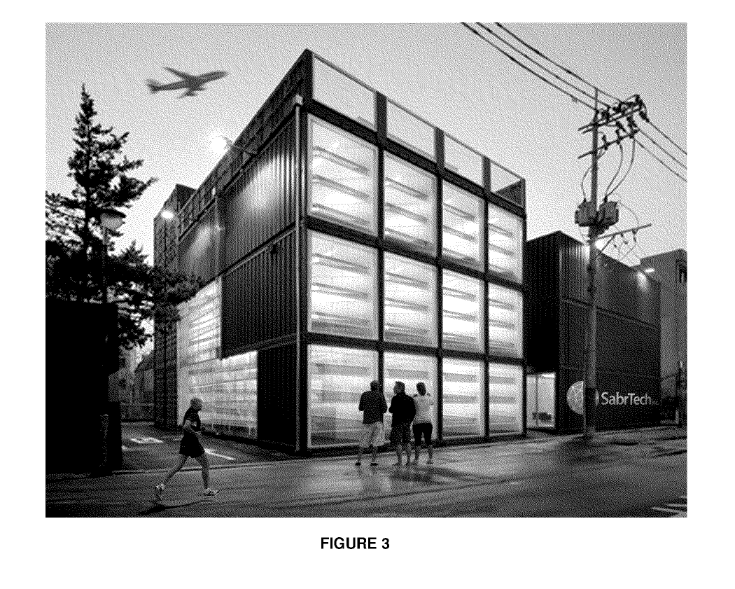

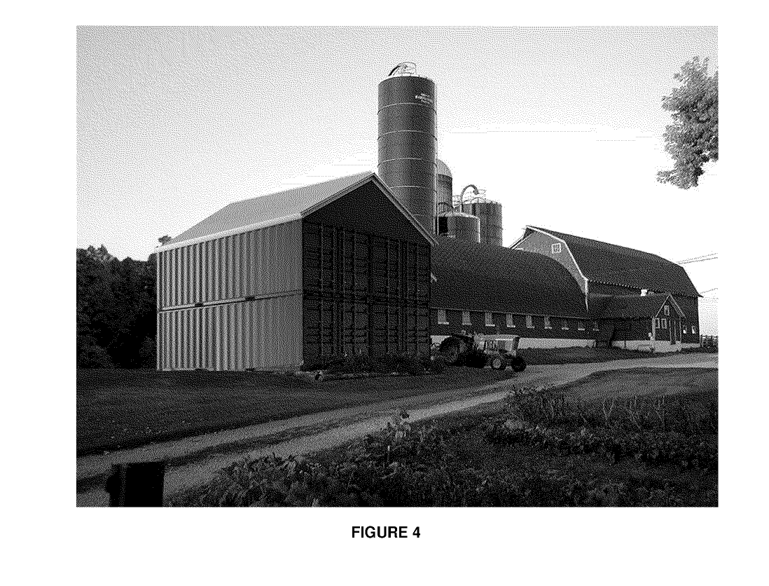

Turning to FIGS. 3 and 4, particular embodiments of modular algal aquaculture systems are illustrated. In these embodiments, multiple modular algal aquaculture systems can be stacked in an urban environment (such as that seen in FIG. 3) or a rural, agricultural setting (as can be seen in FIG. 4).

In this way, multiple modular algal aquaculture components can be interconnected to provide a scalable and stackable system, wherein the supply headers for multiple modular algal aquaculture components can be in fluid communication with one another and similarly the return headers for multiple modular algal aquaculture components can be in fluid communication with one another. In other embodiments, it is contemplated that the return header for a first modular algal aquaculture component can be in fluid communication with the supply header of a second modular algal aquaculture component, and so on. Moreover and as discussed earlier, it is contemplated that each of the multiple algal aquaculture components in a system can be in fluid communication with a common system reservoir, and in other embodiments it is contemplated each of the algal aquaculture components has its own dedicated reservoir, among other arrangements that will be readily understood by the skilled person.

Based on the foregoing, it will be readily understood that the present invention can provide a algal cultivation system that can be scalable, can be installed in and customized for a wide variety of climates and environments, can be manufactured from corrosion resistant materials, can be installed in any geographic or topographic environment, can use any suitable light source, requires minimal power consumption, and is of generally simple design that can be easily manufactured, maintained, operated and repaired.

It is obvious that the foregoing embodiments of the invention are examples and can be varied in many ways. Such present or future variations are not to be regarded as a departure from the spirit and scope of the invention and all such modifications as would be obvious to one skilled in the art are intended to be included within the scope of the following claims.

1. A modular algal aquaculture component comprising:
a cultivation surface having a first end and a second end;
a supply header located adjacent to one of the first end and the second end;
a return header located adjacent to the other of the first end and the second end;
a reservoir, the reservoir in fluid communication with the supply header and the return header; and
a pump, the pump in fluid communication with the reservoir and at least one of the supply header and the return header.

2. The modular algal aquaculture component of claim 1 wherein the cultivation surface is inclined from the horizontal such that water supplied from the supply header flows to the return header under gravity.

3. The modular algal aquaculture component of claim 1 wherein the inclination of the cultivation surface is adjustable such that the cultivation surface can be adjusted between 0° and 90° from horizontal.

4. The modular algal aquaculture component of claim 1 wherein the cultivation surface further comprises an adherence film, the adherence film removably secured to the cultivation surface.

5. The modular algal aquaculture component of claim 1 further comprising a lighting system located adjacent the cultivation surface.

6. The modular algal aquaculture component of claim 1 further comprising a sterilization unit in fluid communication with at least one of the supply header, the return header and the reservoir.

7. The modular algal aquaculture component of claim 1 further comprising a feedstock supply unit in fluid communication with at least one of the supply header, the return header and the reservoir.

8. The modular algal aquaculture component of claim 1 wherein at least one of the supply header and the return header is an open sluiceway.

9. The modular algal aquaculture component of claim 1 wherein the cultivation surface is an open raceway.

10. The modular algal aquaculture component of claim 1 further comprising a mechanical harvesting system adapted to scrape algal growth from the cultivation surface.

11. The modular algal aquaculture component of claim 1 wherein the cultivation surface has a stippled surface.

12. The modular algal aquaculture component of claim 1 wherein at least one of the supply header and the return header further comprises at least one orifice.

13. A modular algal aquaculture system comprising:
a plurality of modular algal aquaculture components, each of the modular algal aquaculture components having: a cultivation surface having a first end and a second end;
a supply header located adjacent to one of the first end and the second end;
a return header located adjacent to the other of the first end and the second end;
a reservoir, the reservoir in fluid communication with the supply header and the return header; and
a pump, the pump in fluid communication with the reservoir and at least one of the supply header and the return header
wherein the return header of a first modular algal aquaculture component fluidly communicates with at least one of the return header and the supply header of a second modular algal aquaculture component and wherein the supply header of a first modular algal aquaculture component fluidly communicates with at least one of the return header and the supply header of a second modular algal aquaculture component.

14. The modular algal aquaculture system of claim 13 wherein the reservoir of a first modular algal aquaculture component is in fluid communication with the reservoir of a second modular algal aquaculture component.

15. The modular algal aquaculture system of claim 13 wherein the reservoir of a first modular algal aquaculture component and the reservoir of a second modular algal aquaculture component are a common system reservoir.

16. The modular algal aquaculture system of claim 13 wherein at least one of the supply header and the return header further comprises at least one orifice.

17. A method of cultivating algae, comprising:
inoculating a cultivation surface with an algal strain;
providing water to the cultivation surface from a reservoir by way of a supply header;
returning water from the cultivation surface to the reservoir by way of a return header; and
harvesting cultivated algae from the cultivation surface using a harvesting system.
